(19) United States
(12) Patent Application Publication
(10) Pub. No.: US 2023/0355825 A1
(43) Pub. Date: Nov. 9, 2023

(54) FRAGRANCE MACHINE

(71) Applicant: Guangdong Yeniu Technology Co., Ltd., Dongguan (CN)

(72) Inventor: Jun Long, Nanchong (CN)

(21) Appl. No.: 18/346,204

(22) Filed: Jul. 1, 2023

(30) Foreign Application Priority Data
Jun. 1, 2023 (CN) 202321378306.1

(51) Int. Cl. A61L 9/14; F24F 8/50

(52) U.S. Cl. CPC A61L 9/14; F24F 8/50; A61L 2209/134

(57) ABSTRACT

The present invention discloses a fragrance machine, comprising: a driving component and an atomizing component are mounted in a housing; the driving component comprises an air pump and a power supply which are arranged in the housing; the air pump is mounted in a cavity jointly defined by a cover body and a mounting seat; the atomizing component comprises an atomizing pipe, an atomizing nozzle, a connecting pipe and a liquid storage bottle. The present application has a good atomization effect, and the noise generated by the air pump during operation can be effectively blocked, so that the noise during operation of the fragrance machine is very low.

### CROSS REFERENCE TO RELATED APPLICATION

This patent application claims the benefit and priority of Chinese Patent Application No. 2023213783061 filed on Jun. 1, 2023, the disclosure of which is incorporated by reference herein in its entirety as part of the present application

### TECHNICAL FIELD

The present invention relates to the field of household electric appliances, and more particularly to a fragrance machine.

### BACKGROUND

The fragrance machine mainly atomizes the liquid fragrance into gas and then emits the gas into the air. There are many types of fragrance machines on the market. One fragrance machine uses a high-speed airflow to disperse the liquid into an atomized state, and this mode has better atomizing effect than other atomizing modes and may not waste the fragrance liquid.

However, the fragrance machine in the above form has a complex structure, and because it uses the high-speed airflow injection mode, the noise generated by the operation of an air pump is large. In addition, the existing fragrance machine is also very inconvenient in supplementing the fragrance liquid in the process of use.

Therefore, how to provide a fragrance machine capable of solving the above problems is an urgent problem for those skilled in the art.

### SUMMARY

In view of this, the present invention provides a fragrance machine.

To achieve the above purpose, the following technical solution is adopted in the present invention:

A fragrance machine comprises:

a housing, wherein a limiting pipe and a mounting seat are embedded inside the housing, a lower end of the limiting pipe is integrally formed with a cover body, and the cover body is buckled on the mounting seat;

a driving component, wherein the driving component comprises an air pump and a power supply, the air pump is mounted in a cavity jointly defined by the cover body and the mounting seat, and the power supply is limited in the housing and is electrically connected with the air pump;

an atomizing component, wherein the atomizing component comprises an atomizing pipe, an atomizing nozzle, a connecting pipe and a liquid storage bottle; one end of the atomizing pipe is opened, and the other end is closed; the atomizing pipe can be embedded into the limiting pipe; the closed end of the atomizing pipe is provided with a mist outlet; an air inlet channel is integrally formed inside the atomizing pipe; one end of the air inlet channel can be communicated with the air pump through an air path; the other end of the air inlet channel penetrates through the closed end of the atomizing pipe and then is fixed and communicated with the atomizing nozzle; the atomizing nozzle is provided with an air spray hole communicated with the air inlet channel; one end of the connecting pipe is fixed with the atomizing nozzle; a side wall of the connecting pipe is provided with a liquid spray hole; a spraying direction of the air spray hole is perpendicular to a spraying direction of the liquid spray hole; a hole centerline of the air spray hole is coplanar with a hole centerline of the liquid spray hole; the liquid spray hole is arranged to point to the hole centerline of the air spray hole; the liquid storage bottle can be embedded into the limiting pipe; an open end of the liquid storage bottle is detachably connected with the closed end of the atomizing pipe; and the mist outlet, the atomizing nozzle and the connecting pipe are limited in the liquid storage bottle.

It can be known from the above technical solution that compared with the prior art, the present invention discloses and provides a fragrance machine. The air pump of the present invention is mounted in the cavity jointly defined by the cover body and the mounting seat, and the cover body and the mounting seat are limited in the housing. Therefore, the noise generated by the air pump during operation can be effectively blocked, and the design makes the noise during operation of the fragrance machine very low. The atomizing component in the present application is detachably connected with the limiting pipe. When there is less fragrance liquid in the liquid storage bottle, the atomizing pipe and the liquid storage bottle can be removed for liquid replenishment. In the present application, the air spray hole and the liquid spray hole are used. The air spray hole disperses the fragrance liquid sprayed at the liquid spray hole into an atomized state. The atomization mode is simple and reliable, and has good atomization effect.

Preferably, the fragrance machine further comprises a panel, a bottom plate, a connecting seat, a control circuit board and an end cover; the housing is tubular and an upper end and a lower end of the housing are blocked with the panel and the bottom plate respectively; the connecting seat is embedded in the housing; an upper end of the connecting seat is fixed with the panel; a lower end of the connecting seat is fixed with the control circuit board; the control circuit board is electrically integrated with a charging module, a timing module, atmosphere lights and a switch; the charging module, the timing module, the atmosphere lights and the switch are electrically connected with the power supply; the open end of the atomizing pipe is fixedly embedded with the end cover which can transmit light; the end cover is provided with a mist outlet hole and a pipe body; the pipe body is located in the atomizing pipe; the mist outlet hole is limited at an inner side of the pipe body; the panel is provided with a first conceding hole; the connecting seat is provided with a second conceding hole; the control circuit board is provided with a third conceding hole; the end cover can be embedded in the first conceding hole and the second conceding hole; the atomizing pipe can be embedded in the second conceding hole and the third conceding hole; a plurality of atmosphere lights are evenly arranged along the circumference direction of the atomizing pipe; the connecting seat is provided with a through hole for conceding the switch; the switch is arranged in the through hole; the panel is provided with a pressing touch position; and the pressing touch position is opposite to the position of the switch. The fragrance machine has a timing function; the fragrance machine is rechargeable and convenient to carry; and after the atmosphere lights are turned on, on the one hand, the beauty of the fragrance machine is improved, and on the other hand, the fragrance machine can serve as a small night light.

Preferably, the end cover is rotatably connected with a lifting handle, the end cover is provided with a groove, and the lifting handle can be limited into the groove. The design is convenient for taking out the atomizing pipe and the liquid storage bottle from the limiting pipe, and meanwhile, the lifting handle can be hidden in the groove when the lifting handle is not used.

Preferably, a charging interface electrically connected with the charging module is fixed on the control circuit board, a side wall of the housing is provided with a mounting hole, and the charging interface is embedded in the mounting hole. The power supply can be charged through the charging interface.

Preferably, an outer side wall of the limiting pipe is provided with a clamping portion, and the power supply is clamped on the clamping portion. The power supply can be reliably fixed to the limiting pipe.

Preferably, a side wall of the atomizing pipe is integrally formed with a bulge; a hole is formed in the bulge and a rubber pipe is embedded on the bulge; the rubber pipe is communicated with the air inlet channel; a pipe wall of the limiting pipe is provided with a vent hole; an inner side wall of the limiting pipe is provided with a limiting chute; the vent hole is arranged at a groove bottom wall of the limiting chute; the bulge can be embedded into the limiting chute; one end of rubber pipe away from the atomizing pipe can be closely abutted against the groove bottom wall of the limiting chute; one end of the rubber pipe away from the atomizing pipe can be directly opposite to and communicated with the vent hole; an outer side wall of the limiting pipe is fixed with a first inserting pipe communicated with the vent hole; and the first inserting pipe is communicated with the air pump through the air path. The atomizing nozzle can be reliably communicated with the air pump through the air path.

Preferably, a second inserting pipe is fixed on the cover body; both ends of the second inserting pipe are arranged on both sides of the cover body respectively; an air outlet end of the air pump is connected with one end of second inserting pipe through a first air pipe; and the other end of the second inserting pipe is connected with the first inserting pipe through a second air pipe. The air pump can be reliably communicated with the first inserting pipe.

Preferably, the atomizing component further comprises a hose; one end of the hose is sleeved on the connecting pipe; and the hose can come into contact with a bottle bottom of the liquid storage bottle. When there is less fragrance liquid in the liquid storage bottle, the connecting pipe can still extract the fragrance liquid.

Preferably, a mounting block is integrally formed on the connecting pipe; one side surface of the mounting block coincides with the hole centerline of the air spray hole, and the side surface is provided with a splayed open slot; a small open end of the open slot is away from the atomizing nozzle; a diversion block is integrally formed centrally at a groove bottom wall of the open slot; and the diversion block is provided with the liquid spray hole. The design can improve the atomization effect of the fragrance machine.

### DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely embodiments of the present invention, and for those ordinary skilled in the art, other drawings can also be obtained according to the drawings without contributing creative labor.

FIG. 1 is an overall axonometric view of a fragrance machine;

FIG. 2 is an exposed axonometric view of a fragrance machine;

FIG. 3 is an amplified schematic diagram of A in FIG. 2;

FIG. 4 is an amplified schematic diagram of B in FIG. 2;

FIG. 5 is a partial axonometric view 1 of a fragrance machine;

FIG. 6 is a partial axonometric view 2 of a fragrance machine;

FIG. 7 is a partial axonometric view 3 of a fragrance machine;

FIG. 8 is an axonometric view of an end cover in a fragrance machine;

FIG. 9 is an axonometric view 1 of an atomizing pipe in a fragrance machine;

FIG. 10 is an axonometric view 2 of an atomizing pipe in a fragrance machine;

FIG. 11 is a partial axonometric view 4 of a fragrance machine.

In the figures:

01 housing; 010 mounting hole; 02 limiting pipe; 020 clamping portion; 021 vent hole; 022 limiting chute; 03 mounting seat; 04 cover body; 05 air pump; 06 power supply; 07 atomizing pipe; 070 mist outlet; 071 air inlet channel; 072 bulge; 08 atomizing nozzle; 080 air spray hole; 09 connecting pipe; 090 liquid spray hole; 091 mounting block; 092 open slot; 093 diversion block; 10 liquid storage bottle; 11 hose; 12 panel; 120 first conceding hole; 121 pressing touch position; 13 bottom plate; 14 connecting seat; 140 second conceding hole; 141 through hole; 15 control circuit board; 150 third conceding hole; 16 atmosphere light; 17 switch; 18 end cover; 180 mist outlet hole; 181 pipe body; 182 groove; 19 lifting handle; 20 charging interface; 21 rubber pipe; 22 first inserting pipe; 23 second inserting pipe; 24 first air pipe; 25 second air pipe.

### DETAILED DESCRIPTION

The technical solutions in embodiments of the present invention will be clearly and fully described below. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

The present invention discloses a fragrance machine. An air pump 05 of the present invention is mounted in a cavity jointly defined by a cover body 04 and the mounting seat 03, and the cover body 04 and the mounting seat 03 are limited in a housing 01. Therefore, the noise generated by the air pump 05 during operation can be effectively blocked, and the design makes the noise of the fragrance machine very low during operation. An atomizing component in the present application is detachably connected with a limiting pipe 02. When there is less fragrance liquid in a liquid storage bottle 10, an atomizing pipe 07 and the liquid storage bottle 10 can be removed for liquid replenishment. In the present application, an air spray hole 080 and a liquid spray hole 090 are used. The air spray hole 080 disperses the fragrance liquid sprayed at the liquid spray hole 090 into an atomized state. The atomization mode is simple and reliable, and has good atomization effect. An end cover 18 is provided with a lifting handle 19, and the design is convenient for a user to take out the atomizing pipe 07 and the liquid storage bottle 10 from the limiting pipe 02.

### EMBODIMENTS

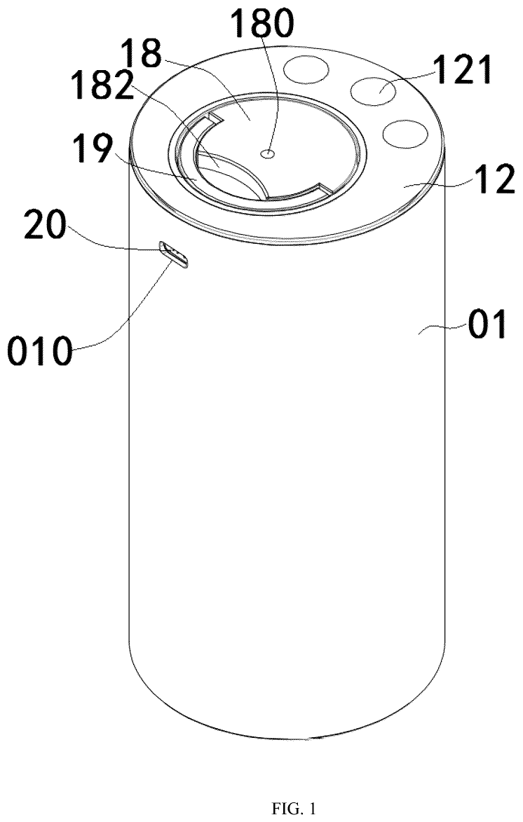

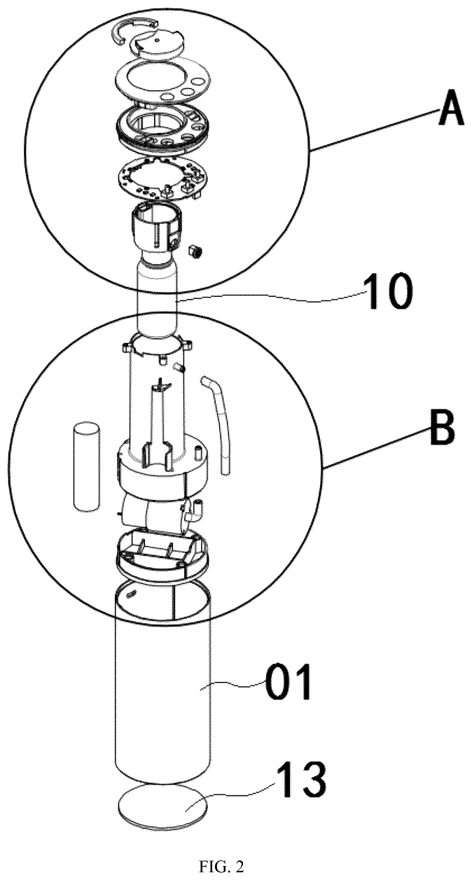

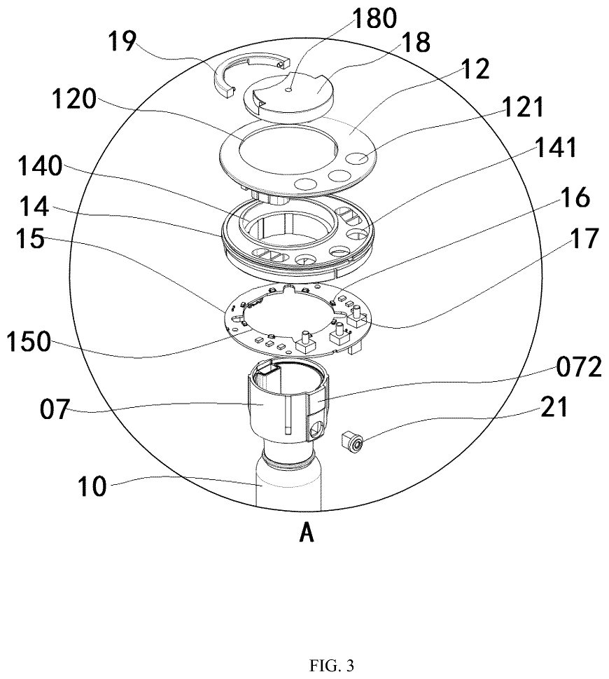

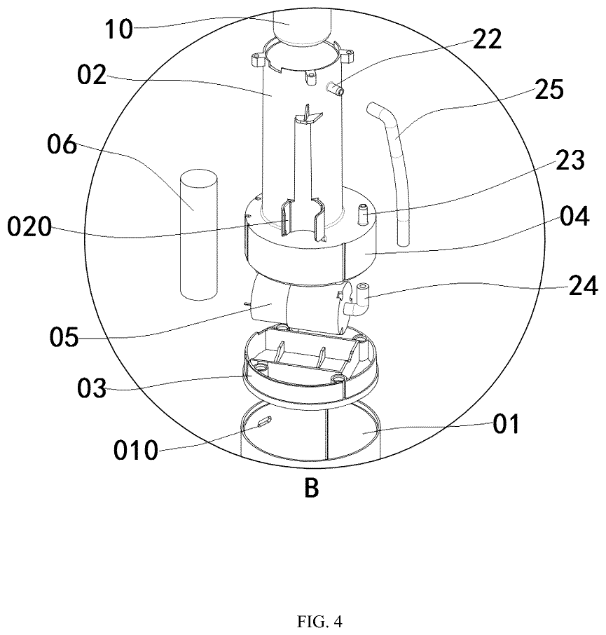

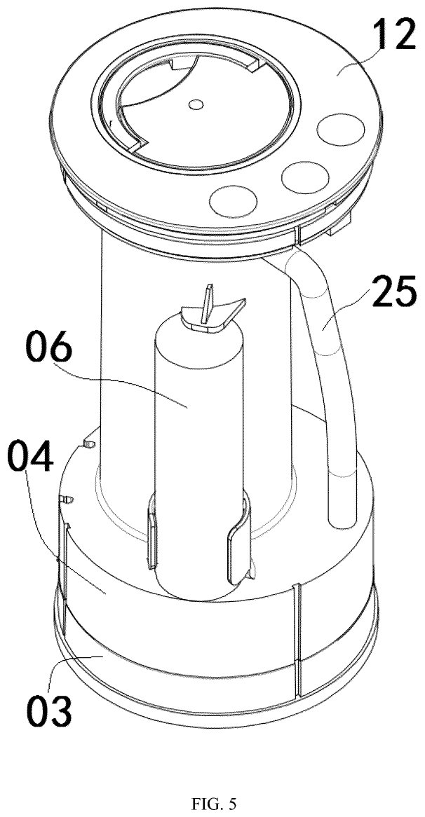

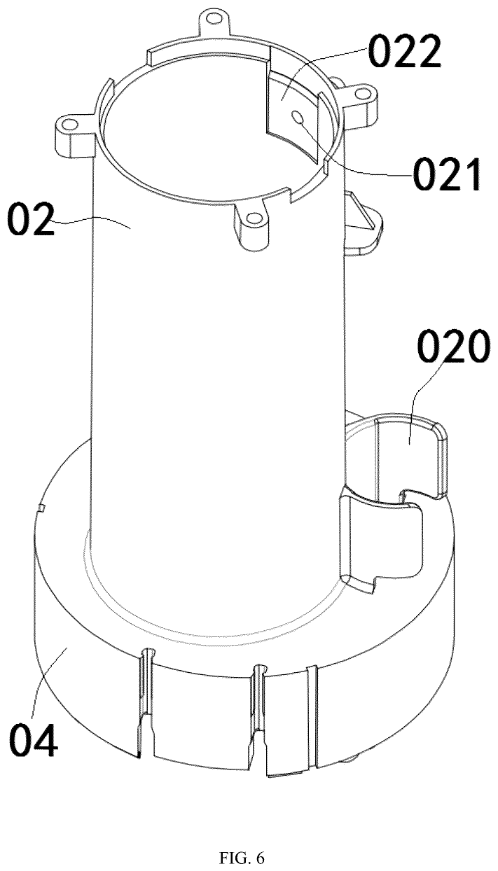

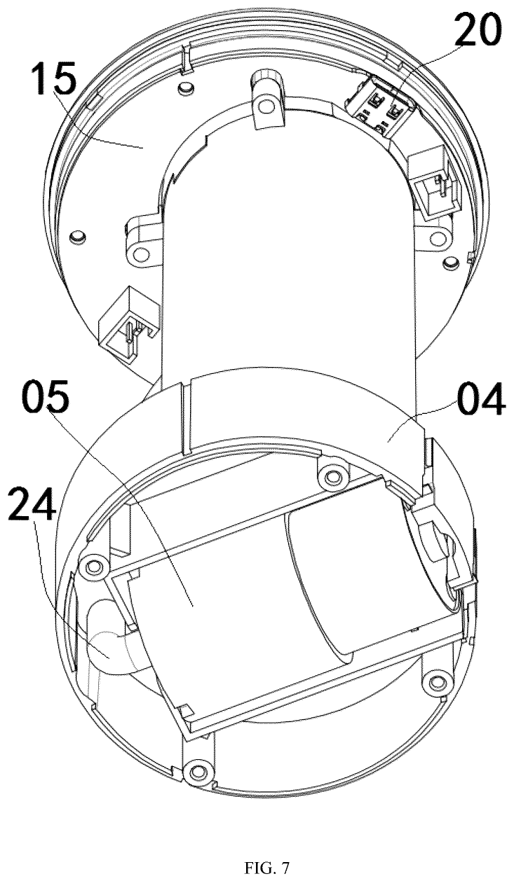

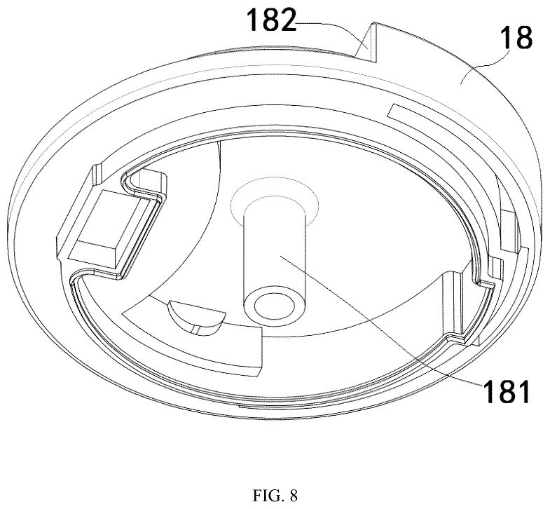

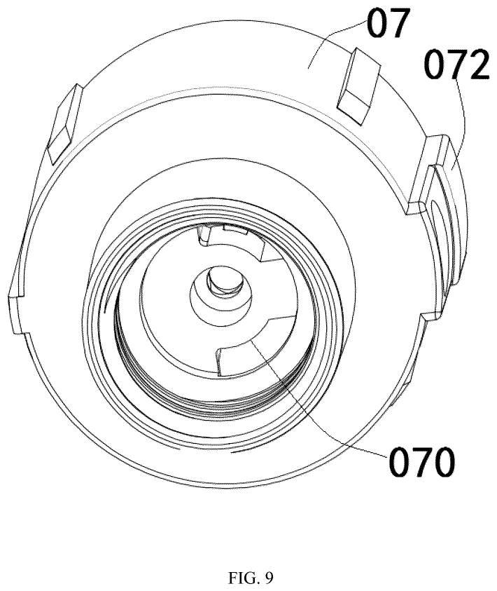

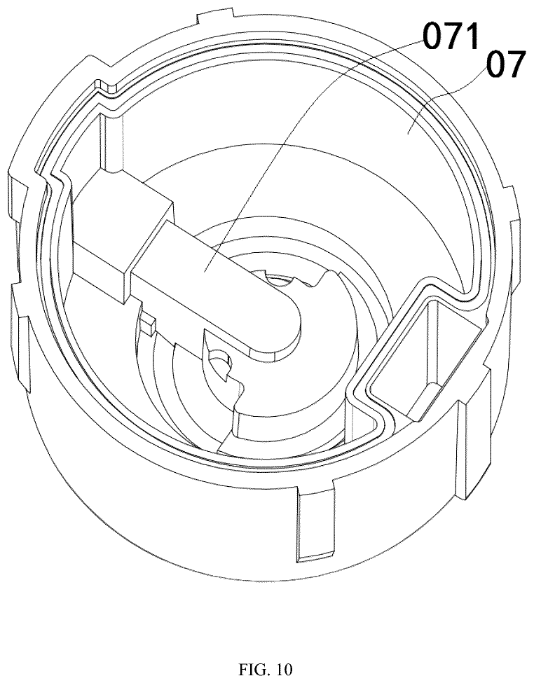

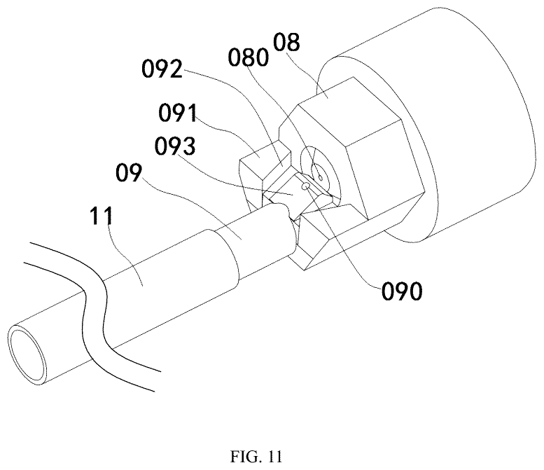

By referring to FIGS. 1-11 which are overall and partial structural schematic diagrams of an embodiment of the present invention, the present invention specifically discloses a fragrance machine which comprises:

a tubular housing 01 which is open at an upper end and a lower end, wherein a limiting pipe 02 and a mounting seat 03 are embedded inside the housing 01; a pipe centerline of the limiting pipe 02 is parallel to the axis line of the housing 01; a lower end of the limiting pipe 02 is integrally formed with a cover body 04; the cover body 04 blocks the lower end of the limiting pipe 02; the cover body 04 is buckled on the mounting seat 03; and a cavity is jointly defined by the cover body 04 and the mounting seat 03;

a driving component, wherein the driving component comprises an air pump 05 and a power supply 06; the air pump 05 is embedded in the cavity jointly defined by the cover body 04 and the mounting seat 03; the air pump 05 is mounted in the cavity; the cover body 04 and the mounting seat 03 can further block the noise generated during the operation of the air pump 05; the power supply 06 is limited in the housing 01 and is electrically connected with the air pump 05; and the power supply 06 in the present application is cylindrical and is a rechargeable lithium battery;

an atomizing component, wherein the atomizing component comprises an atomizing pipe 07, an atomizing nozzle 08, a connecting pipe 09 and a liquid storage bottle 10; one end of the atomizing pipe 07 is opened, and the other end is closed; the atomizing pipe can be embedded into the limiting pipe 02; the closed end of the atomizing pipe 07 is provided with a mist outlet 070; an air inlet channel 071 is integrally formed inside the atomizing pipe 07; one end of the air inlet channel 071 can be communicated with the air pump 05 through an air path; the other end of the air inlet channel 071 penetrates through the closed end of the atomizing pipe 07 and then is fixed and communicated with the atomizing nozzle 08; the atomizing nozzle 08 is provided with an air spray hole 080 communicated with the air inlet channel 071; one end of the connecting pipe 09 is fixed with the atomizing nozzle 08; a side wall of the connecting pipe 09 is provided with a liquid spray hole 090; a spraying direction of the air spray hole 080 is perpendicular to a spraying direction of the liquid spray hole 090; a hole centerline of the air spray hole 080 is coplanar with a hole centerline of the liquid spray hole 090; and the liquid spray hole 090 is arranged to point to the hole centerline of the air spray hole 080;

the liquid storage bottle 10 can be embedded into the limiting pipe 02; a rifled pipe is arranged at an outer side of the closed end of the atomizing pipe 07; an open end of the liquid storage bottle 10 is screwed with the rifled pipe; the mist outlet 070, the atomizing nozzle 08, the connecting pipe 09 and the hose 11 are limited in the liquid storage bottle 10; the air spray hole 080 sprays gas at high speed; negative pressure is generated at the position of the liquid spray hole 090; the connecting pipe 09 absorbs the fragrance liquid in the liquid storage bottle 10 and sprays out the fragrance liquid from the liquid spray hole 090; the high-speed gas sprayed from the air spray hole 080 disperses the fragrance liquid sprayed from the liquid spray hole 090 into atomized gas; and the atomized gas is discharged from the mist outlet 070.

Further specifically, the fragrance machine further comprises a panel 12, a bottom plate 13, a connecting seat 14, a control circuit board 15 and an end cover 18.

An upper end and a lower end of the housing 01 are blocked with the panel 12 and the bottom plate 13 respectively; the panel 12 is made of light transmission material; the connecting seat 14 is embedded in the housing 01; an upper end of the connecting seat 14 is fixed with the panel 12; a lower end of the connecting seat 14 is fixed with the control circuit board 15; the control circuit board 15 is electrically integrated with a charging module, a timing module, atmosphere lights 16 and switches 17; and the charging module, the timing module, the atmosphere lights 16 and the switches 17 are electrically connected with the power supply 06. Three switches 17 are arranged in the present embodiment. The first switch 17 controls the air pump 05, the second switch 17 controls the atmosphere lights 16, and the third switch 17 is responsible for timing.

The open end of the atomizing pipe 07 is fixedly embedded with the end cover 18 which can transmit light; the end cover 18 is provided with a mist outlet hole 180 and a pipe body 181; the pipe body 181 is located in the atomizing pipe 07; and the mist outlet hole 180 is limited at an inner side of the pipe body 181. The pipe body 181 is designed to prevent the fragrance liquid in the liquid storage bottle 10 from being discharged from the mist outlet hole 180 when the housing 01 is obliquely arranged.

The panel 12 is provided with a first conceding hole 120; the connecting seat 14 is provided with a second conceding hole 140; and the control circuit board 15 is provided with a third conceding hole 150.

The end cover 18 can be embedded in the first conceding hole 120 and the second conceding hole 140; the atomizing pipe 07 can be embedded in the second conceding hole 140 and the third conceding hole 150, i.e., the end cover 18 and the atomizing pipe 07 are embedded in the second conceding hole 140; a plurality of atmosphere lights 16 are evenly arranged along the circumference direction of the atomizing pipe 07; and an illumination direction of the atmosphere lights 16 is away from the control circuit board 15 and the bottom board 13.

The connecting seat 14 is provided with a through hole 141 for conceding the switches 17; the switches 17 are arranged in the through hole 141; the panel 12 is provided with a pressing touch position 121; and the pressing touch position 121 is opposite to the positions of the switches 17. In the present embodiment, the switches 17 are a push-button switches; the keys of the switches 17 are in contact with the pressing touch position 121; the pressing touch position 121 on the panel 12 can be pressed to deform; and when the pressing touch position 121 is pressed down, the keys of the switches 17 also move, thereby triggering the switches 17.

The connecting seat 14 is also provided with a light transmission hole; the control circuit board 15 is electrically integrated with an indicator light; and when the indicator light is opposite to the position of the light transmission hole, the indicator light can prompt the electric quantity of the power supply 06 and the air outlet speed of the air pump 05, i.e., the outlet amount of mist.

The end cover 18 is rotatably connected with a semi-arc lifting handle 19; the end cover 18 is provided with a groove 182; and when the lifting handle 19 is not used, the lifting handle 19 is rotated to limit the lifting handle into the groove 182.

A charging interface 20 electrically connected with the charging module is fixed on the control circuit board 15, a side wall of the housing 01 is provided with a mounting hole 010, and the charging interface 20 is embedded in the mounting hole 010.

An outer side wall of the limiting pipe 02 is provided with a clamping portion 020, and the power supply 06 is clamped on the clamping portion 020.

A side wall of the atomizing pipe 07 is integrally formed with a bulge 072; a hole is formed in the bulge 072 and a rubber pipe 21 is embedded on the bulge; the rubber pipe 21 is communicated with the air inlet channel 071; a pipe wall of the limiting pipe 02 is provided with a vent hole 021; an inner side wall of the limiting pipe 02 is provided with a limiting chute 022; the vent hole 021 is arranged at a groove bottom wall of the limiting chute 022; the bulge 072 can be embedded into the limiting chute 022; one end of rubber pipe 21 away from the atomizing pipe 07 can be closely abutted against the groove bottom wall of the limiting chute 022; one end of the rubber pipe 21 away from the atomizing pipe 07 can be directly opposite to and communicated with the vent hole 021; an outer side wall of the limiting pipe 02 is fixed with a first inserting pipe 22 communicated with the vent hole 021; and the first inserting pipe 22 is communicated with the air pump 05 through the air path. The main purpose of the design of the bulge 072 and the limiting chute 022 is to limit the atomizing pipe 07 to ensure that the rubber pipe 21 can be accurately opposite to and communicated with the vent hole 021.

A second inserting pipe 23 is fixed on the cover body 04; both ends of the second inserting pipe 23 are arranged on both sides of the cover body 04 respectively; an air outlet end of the air pump 05 is connected with one end of second inserting pipe 23 through a first air pipe 24; and the other end of the second inserting pipe 23 is connected with the first inserting pipe 22 through a second air pipe 25.

The atomizing component further comprises a hose 11; one end of the hose 11 is sleeved on the connecting pipe 09; and the hose 11 can come into contact with a bottle bottom of the liquid storage bottle 10. The design ensures that the connecting pipe 09 can still extract the fragrance liquid in the liquid storage bottle 10 when there is less fragrance liquid in the liquid storage bottle 10.

A mounting block 091 is integrally formed on the connecting pipe 09; one side surface of the mounting block 091 coincides with the hole centerline of the air spray hole 080, and the side surface is provided with a splayed open slot 092; a small open end of the open slot 092 is away from the atomizing nozzle 08; an arc diversion block 093 is integrally formed centrally at a groove bottom wall of the open slot 092; and an arc surface of the diversion block 093 is provided with the liquid spray hole 090. The design can improve the atomization effect of the fragrance machine.

When the fragrance machine is operated, the air spray hole 080 communicated with the air pump 05 through the air path sprays high-speed gas into the liquid storage bottle 10; negative pressure is generated at the liquid spray hole 090, and the fragrance liquid in the liquid storage bottle 10 is extracted; the fragrance liquid flows out from the liquid spray hole 090 and then is dispersed by the high-speed airflow sprayed by the air spray hole 080 into an atomized state; and the atomized fragrance liquid is discharged from the mist outlet 070, the atomizing pipe 07, the pipe body 181 and the mist outlet hole 180 successively.

When the fragrance liquid needs to be supplemented into the liquid storage bottle 10, the user pulls the lifting handle 19 to pull out the atomizing pipe 07 and the liquid storage bottle 10 from the limiting pipe 02, and then the liquid storage bottle 10 and the atomizing pipe 07 are separated. After the liquid storage bottle 10 is supplemented with the liquid, the liquid storage bottle 10 is connected with the atomizing pipe 07, then the bulge 072 is aligned with the limiting chute 022, and finally the atomizing pipe 07 and the liquid storage bottle 10 are inserted into the limiting pipe 02 again.

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A fragrance machine, comprising:
a housing (01), wherein a limiting pipe (02) and a mounting seat (03) are embedded inside the housing (01), a lower end of the limiting pipe (02) is integrally formed with a cover body (04), and the cover body (04) is buckled on the mounting seat (03);
a driving component, wherein the driving component comprises an air pump (05) and a power supply (06), the air pump (05) is mounted in a cavity jointly defined by the cover body (04) and the mounting seat (03), and the power supply (06) is limited in the housing (01) and is electrically connected with the air pump (05);
an atomizing component, wherein the atomizing component comprises an atomizing pipe (07), an atomizing nozzle (08), a connecting pipe (09) and a liquid storage bottle (10); one end of the atomizing pipe (07) is opened, and the other end is closed; the atomizing pipe can be embedded into the limiting pipe (02); the closed end of the atomizing pipe (07) is provided with a mist outlet (070); an air inlet channel (071) is integrally formed inside the atomizing pipe (07); one end of the air inlet channel (071) can be communicated with the air pump (05) through an air path; the other end of the air inlet channel (071) penetrates through the closed end of the atomizing pipe (07) and then is fixed and communicated with the atomizing nozzle (08); the atomizing nozzle (08) is provided with an air spray hole (080) communicated with the air inlet channel (071); one end of the connecting pipe (09) is fixed with the atomizing nozzle (08); a side wall of the connecting pipe (09) is provided with a liquid spray hole (090); a spraying direction of the air spray hole (080) is perpendicular to a spraying direction of the liquid spray hole (090); a hole centerline of the air spray hole (080) is coplanar with a hole centerline of the liquid spray hole (090); the liquid spray hole (090) is arranged to point to the hole centerline of the air spray hole (080); the liquid storage bottle (10) can be embedded into the limiting pipe (02); an open end of the liquid storage bottle (10) is detachably connected with the closed end of the atomizing pipe (07); and the mist outlet (070), the atomizing nozzle (08) and the connecting pipe (09) are limited in the liquid storage bottle (10).

2. The fragrance machine according to claim 1, further comprising a panel (12), a bottom plate (13), a connecting seat (14), a control circuit board (15) and an end cover (18); the housing (01) is tubular and an upper end and a lower end of the housing are blocked with the panel (12) and the bottom plate (13) respectively; the connecting seat (14) is embedded in the housing (01); an upper end of the connecting seat (14) is fixed with the panel (12); a lower end of the connecting seat (14) is fixed with the control circuit board (15); the control circuit board (15) is electrically integrated with a charging module, a timing module, atmosphere lights (16) and a switch (17); the charging module, the timing module, the atmosphere lights (16) and the switch (17) are electrically connected with the power supply (06); the open end of the atomizing pipe (07) is fixedly embedded with the end cover (18) which can transmit light; the end cover (18) is provided with a mist outlet hole (180) and a pipe body (181); the pipe body (181) is located in the atomizing pipe (07); the mist outlet hole (180) is limited at an inner side of the pipe body (181); the panel (12) is provided with a first conceding hole (120); the connecting seat (14) is provided with a second conceding hole (140); the control circuit board (15) is provided with a third conceding hole (150); the end cover (18) can be embedded in the first conceding hole (120) and the second conceding hole (140); the atomizing pipe (07) can be embedded in the second conceding hole (140) and the third conceding hole (150); a plurality of atmosphere lights (16) are evenly arranged along the circumference direction of the atomizing pipe (07); the connecting seat (14) is provided with a through hole (141) for conceding the switch (17); the switch (17) is arranged in the through hole (141); the panel (12) is provided with a pressing touch position (121); and the pressing touch position (121) is opposite to the position of the switch (17).

3. The fragrance machine according to claim 2, wherein the end cover (18) is rotatably connected with a lifting handle (19), the end cover (18) is provided with a groove (182), and the lifting handle (19) can be limited into the groove (182).

4. The fragrance machine according to claim 2, wherein a charging interface (20) electrically connected with the charging module is fixed on the control circuit board (15), a side wall of the housing (01) is provided with a mounting hole (010), and the charging interface (20) is embedded in the mounting hole (010).

5. The fragrance machine according to claim 1, wherein an outer side wall of the limiting pipe (20) is provided with a clamping portion (020), and the power supply (06) is clamped on the clamping portion (020).

6. The fragrance machine according to claim 1, wherein a side wall of the atomizing pipe (07) is integrally formed with a bulge (072); a hole is formed in the bulge (072) and a rubber pipe (21) is embedded on the bulge; the rubber pipe (21) is communicated with the air inlet channel (071); a pipe wall of the limiting pipe (02) is provided with a vent hole (021); an inner side wall of the limiting pipe (02) is provided with a limiting chute (022); the vent hole (021) is arranged at a groove bottom wall of the limiting chute (022); the bulge (072) can be embedded into the limiting chute (022); one end of rubber pipe (21) away from the atomizing pipe (07) can be closely abutted against the groove bottom wall of the limiting chute (022); one end of the rubber pipe (21) away from the atomizing pipe (07) can be directly opposite to and communicated with the vent hole (021); an outer side wall of the limiting pipe (02) is fixed with a first inserting pipe (22) communicated with the vent hole (021); and the first inserting pipe (22) is communicated with the air pump (05) through the air path.

7. The fragrance machine according to claim 6, wherein a second inserting pipe (23) is fixed on the cover body (04); both ends of the second inserting pipe (23) are arranged on both sides of the cover body (04) respectively; an air outlet end of the air pump (05) is connected with one end of second inserting pipe (23) through a first air pipe (24); and the other end of the second inserting pipe (23) is connected with the first inserting pipe (22) through a second air pipe (25).

8. The fragrance machine according to claim 1, wherein the atomizing component further comprises a hose (11); one end of the hose (11) is sleeved on the connecting pipe (09); and the hose (11) can come into contact with a bottle bottom of the liquid storage bottle (10).

9. The fragrance machine according to claim 1, wherein a mounting block (091) is integrally formed on the connecting pipe (09); one side surface of the mounting block (091) coincides with the hole centerline of the air spray hole (080), and the side surface is provided with a splayed open slot (092); a small open end of the open slot (092) is away from the atomizing nozzle (08); a diversion block (093) is integrally formed centrally at a groove bottom wall of the open slot (092); and the diversion block (093) is provided with the liquid spray hole (090).
